(19) United States
(12) Patent Application Publication
(10) Pub. No.: US 2023/0309948 A1
(43) Pub. Date: Oct. 5, 2023

(54) DATA TRANSMISSION SYSTEMS AND METHODS

(71) Applicant: SHANGHAI UNITED IMAGING HEALTHCARE CO., LTD., Shanghai (CN)

(72) Inventors: Guangxin DING, Shanghai (CN); Zhouyuan FAN, Shanghai (CN); Yifeng JIANG, Shanghai (CN)

(73) Assignee: SHANGHAI UNITED IMAGING HEALTHCARE CO., LTD., Shanghai (CN)

(21) Appl. No.: 18/295,242

(22) Filed: Apr. 3, 2023

(30) Foreign Application Priority Data
Apr. 1, 2022 (CN) 202210339330.8
May 16, 2022 (CN) 202210527145.1

(51) Int. Cl. A61B 6/00; G06T 17/00; G06T 7/73; G16H 10/60

(52) U.S. Cl. CPC A61B 6/56; G06T 17/00; G06T 7/74; G16H 10/60

(57) ABSTRACT

A data transmission system used in a medical device may be provided. The medical device may include a rotating part that rotates during a medical procedure and a stationary part that keeps still during the medical procedure. The data transmission system may include an RF emitter and an RF receiver. The RF emitter may be mounted on one of the rotating part and the stationary part, and the RF receiver may be mounted on the other one of the rotating part and the stationary part. The RF emitter may be configured to generate a target RF signal encoding target data based on a current relative position of the RF emitter with respect to the RF receiver, and transmit the target RF signal to the RF receiver. The RF receiver may be configured to receive the target RF signal and extract the target data from the target RF signal.

### CROSS-REFERENCE TO RELATED APPLICATIONS

This application claims priority to Chinese Patent Application No. 202210339330.8 filed on Apr. 1, 2022 and Chinese Patent Application No. 202210527145.1 filed on May 16, 2022, the entire contents of each of which are hereby incorporated by reference.

### TECHNICAL FIELD

The present disclosure generally relates to the data transmission field, and more particularly, relates to data transmission systems and methods used in a medical device.

### BACKGROUND

A medical device (e.g., a medical imaging device) has been widely used to collect scan data of a subject (e.g., a patient) for disease diagnosis and treatment, etc. As a scanning speed of the medical device increases, a collection speed of the scan data becomes faster and faster. How to accurately and quickly transmit the scan data to a data processing device is vital for improving the efficiency and accuracy of disease diagnosis and treatment.

Thus, it is desired to provide data transmission systems and methods with improved efficiency and accuracy.

### SUMMARY

According to an aspect of the present disclosure, a data transmission system used in a medical device may be provided. The medical device may include a rotating part that rotates during a medical procedure and a stationary part that keeps still during the medical procedure. The data transmission system may include a radio frequency (RF) emitter and an RF receiver. The RF emitter may be mounted on one of the rotating part and the stationary part, and the RF receiver may be mounted on the other one of the rotating part and the stationary part. The RF emitter may be configured to generate a target RF signal encoding target data based on a current relative position of the RF emitter with respect to the RF receiver, and transmit the target RF signal to the RF receiver. The RF receiver may be configured to receive the target RF signal and extract the target data from the target RF signal.

In some embodiments, the RF emitter may be mounted on the rotating part, the RF receiver may be mounted on the stationary part, and the target data may include scan data of a subject collected during the medical procedure.

In some embodiments, the RF receiver may be further configured to transmit the scan data extracted from the target RF signal to an image reconstruction component for reconstructing an image of the subject based on the scan data.

In some embodiments, the RF emitter may include a plurality of emitting units, and the target RF signal may be generated by perform the following operations. An initial RF signal may be generated. The initial RF signal may include a plurality of initial RF sub-signals each of which is generated via one of the plurality of emitting units of the RF emitter. For each of the plurality of initial RF sub-signals, a target RF sub-signal may be generated by adjusting one or more parameters of the initial RF sub-signal based on the current relative position of the RF emitter with respect to the RF receiver. The target RF signal may be generated based on the plurality of target RF sub-signals.

In some embodiments, the one or more parameters may include one or more of a carrier frequency, an encoding mode, a modulation mode, a magnitude, or a phase.

In some embodiments, the adjusting one or more parameters of the initial RF sub-signal based on the current relative position of the RF emitter with respect to the RF receiver may include the following operations. For each of the one or more parameters of the initial RF sub-signal, a relationship between an adjustment coefficient of the parameter and a relative position of the RF emitter with respect to the RF receiver may be obtained. The parameter may be adjusted based on the relationship and the current relative position of the RF emitter with respect to the RF receiver.

In some embodiments, the relationship between an adjustment coefficient of a parameter of the initial RF sub-signal and the relative position may be determined by performing the following operations. An initial relationship between the adjustment coefficient of the parameter and the relative position may be determined. A test result of the initial relationship may be generated by performing a reference medical procedure. The relationship between the adjustment coefficient of the parameter and the relative position may be determined by modifying the initial relationship based on the test result.

In some embodiments, the determining an initial relationship between the adjustment coefficient of the parameter and the relative position may include the following operations. Possible relative positions of the RF emitter with respect to the RF receiver during a rotation period of the rotating part may be determined. For each of the possible relative positions, an impact of at least one of a Doppler effect or a multipath effect corresponding to the possible relative position may be estimated. For each of the possible relative positions, an adjustment value corresponding to the parameter may be determined based on the estimated impact. The initial relationship between the adjustment coefficient of the parameter and the relative position may be determined based on the adjustment value corresponding to each of the possible relative positions.

In some embodiments, the RF receiver may be mounted on the stationary part, an installation location of the RF receiver on the stationary part may be determined by performing the following operations. A three-dimension (3D) model of the medical device may be obtained. The 3D model may include a virtual rotating part, a virtual RF emitter mounted on the virtual rotating part, a virtual stationary part, and a plurality of virtual RF receivers mounted on a plurality of reference installation positions of the virtual stationary part. For each of the plurality of reference installation positions, an evaluation result corresponding to the reference installation position may be determined by simulating a data transmission process between the virtual RF emitter and the virtual RF receiver mounted on the reference install position. The installation location of the RF receiver on the stationary part may be determined based on the evaluation results of the plurality of reference installation positions.

According to another aspect of the present disclosure, another data transmission system used in a medical device may be provided. The medical device may include a rotating part that rotates during a medical procedure and a stationary part that keeps still during the medical procedure. The data transmission system may include an optical source and at least one photoelectric receiver. The optical source may be mounted on one of the rotating part and the stationary part, the at least one photoelectric receiver may be mounted on the other one of the rotating part and the stationary part. The optical source may include optical emitters configured to emit optical signals encoding target data. When the rotating part rotates during the medical procedure, at least one of the optical emitters may move into a detection zone of the at least one photoelectric receiver, and the at least one photoelectric receiver may be configured to detect the optical signals from the at least one optical emitter and extract the target data from the detected optical signals.

In some embodiments, the optical source may be mounted on the rotating part, the at least one photoelectric receiver may be mounted on the stationary part, and the target data may include scan data of a subject collected during the medical procedure.

In some embodiments, the optical source may be mounted on the stationary part, the at least one photoelectric receiver may be mounted on the rotating part, and the target data may include a control instruction that is used to cause one or more components of the rotating part to perform one or more operations.

In some embodiments, the optical source may cover a portion of the rotating part along a circumferential of the rotating part, and the at least one photoelectric receiver may include a plurality of photoelectric receivers. When the rotating part rotates during the medical procedure, the plurality of photoelectric receivers may sequentially detect the optical signals from the optical source.

In some embodiments, the at least one photoelectric receiver may include one photoelectric receiver, and the optical source may cover the rotating part along a circumferential of the rotating part.

In some embodiments, the target data may be divided into a plurality sets of data, the optical source may include a plurality of optical sub-arrays each of which is configured to transmit one set of the plurality sets of data. The at least one photoelectric receiver may include a plurality of photoelectric receivers, when the rotating part rotates during the medical procedure, the optical signals from each of the plurality of optical sub-arrays may be detected by one of the plurality of photoelectric receivers.

In some embodiments, a count of the plurality of optical sub-arrays may be determined according to a transmission speed of the target data.

In some embodiments, the at least one photoelectric receiver may be further configured to transmit the scan data extracted from the detected optical signals to an image reconstruction component for reconstructing an image of the subject based on the scan data.

In some embodiments, the one of the rotating part and the stationary part further may include a modulation driving module configured to drive the optical source to emit the optical signals.

In some embodiments, the at least one photoelectric receiver may further include a modulation and demodulation module configured to modulate and demodulate the detected optical signals to extract the target data.

In some embodiments, the data transmission system may include a second optical source and at least one second photoelectric receiver. The second optical source may be mounted on the other one of the rotating part and the stationary part where the at least one photoelectric receiver is mounted on, and the at least one second photoelectric receiver may be mounted on the one of the rotating part and the stationary part where the optical source is mounted on.

Additional features may be set forth in part in the description which follows, and in part may become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating an exemplary rotating part of the medical device 110 according to some embodiments of the present disclosure;

FIG. 3 is a flowchart illustrating an exemplary process for generating a target RF signal according to some embodiments of the present disclosure;

FIG. 4 is a flowchart illustrating an exemplary process for determining a relationship between an adjustment value of a parameter of an RF signal and a relative position of the RF emitter 121 with respect to the RF receiver 122 according to some embodiments of the present disclosure;

FIG. 5 is a flowchart illustrating an exemplary process for determining an installation location of the RF receiver 122 on the stationary part 112 according to some embodiments of the present disclosure;

FIG. 6 is a schematic diagram illustrating an exemplary medical system according to some embodiments of the present disclosure;

FIG. 7 is a schematic diagram illustrating an exemplary medical system according to some embodiments of the present disclosure;

FIG. 8 is a schematic diagram illustrating an exemplary medical system according to some embodiments of the present disclosure;

FIG. 9 is a schematic diagram illustrating an exemplary medical system 900 according to some embodiments of the present disclosure;

FIG. 10 is a schematic diagram illustrating an exemplary data transmission system 1000 according to some embodiments of the present disclosure;

FIG. 11 is a schematic diagram illustrating an exemplary data transmission system 1100 according to some embodiments of the present disclosure;

FIG. 12 is a schematic diagram illustrating an exemplary data transmission system 1200 in MISO mode according to some embodiments of the present disclosure; and

FIG. 13 is a schematic diagram illustrating an exemplary data transmission system 1300 in MIMO mode according to some embodiments of the present disclosure.

### DETAILED DESCRIPTION

In the following detailed description, numerous specific details may be set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments may be readily apparent to those skilled in the art, and the general principles defined herein may be applied to other embodiments and applications without departing from the spirit and scope of the present disclosure. Thus, the present disclosure may be not limited to the embodiments shown, but to be accorded the widest scope consistent with the claims.

The terminology used herein may be for the purpose of describing particular example embodiments only and may be not intended to be limiting. As used herein, the singular forms “a,” “an,” and “the” may be intended to include the plural forms as well, unless the context clearly indicates otherwise. It may be understood that the terms “system,” “unit,” “module,” and/or “block” used herein are one method to distinguish different components, elements, parts, sections or assemblies of different levels in ascending order. However, the terms may be displaced by another expression if they achieve the same purpose.

The modules (or units, blocks, units) described in the present disclosure may be implemented as software and/or hardware modules and may be stored in any type of non-transitory computer-readable medium or other storage devices. In some embodiments, a software module may be compiled and linked into an executable program. It may be appreciated that software modules may be callable from other modules or from themselves, and/or may be invoked in response to detected events or interrupts. Software modules configured for execution on computing devices may be provided on a computer readable medium or as a digital download (and can be originally stored in a compressed or installable format that requires installation, decompression, or decryption prior to execution). Such software code may be stored, partially or fully, on a memory device of the executing computing device, for execution by the computing device. Software instructions may be embedded in a firmware, such as an EPROM. It may be further appreciated that hardware modules (e.g., circuits) may be included in connected or coupled logic units, such as gates and flip-flops, and/or may be included in programmable units, such as programmable gate arrays or processors. The modules or computing device functionality described herein may be preferably implemented as hardware modules, but may be software modules as well. In general, the modules described herein refer to logical modules that may be combined with other modules or divided into units despite their physical organization or storage.

Certain terminology has been used to describe embodiments of the present disclosure. For example, the terms “one embodiment,” “an embodiment,” and/or “some embodiments” may mean that a particular feature, structure or characteristic described in connection with the embodiment is in at least one embodiment of the present disclosure. Therefore, it is emphasized and should be appreciated that two or more references to “an embodiment” or “one embodiment” or “an alternative embodiment” in various portions of this specification may not necessarily all referring to the same embodiment. Furthermore, the particular features, structures or characteristics may be combined as suitable in one or more embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings may be for the purpose of illustration and description only and may be not intended to limit the scope of the present disclosure.

The flowcharts used in the present disclosure may illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

As used herein, the data transmission systems and methods mainly are used for data transmission between a rotating part and a stationary part of a medical device. For illustration purposes, the data transmission systems and methods for a computed tomography (CT) system are described hereinafter.

Usually, a CT system may include a rotating part and a stationary part. The rotating part may include a radiation source, a detector etc., that are configured to collect scan data and rotate around a subject during a medical procedure. The stationary part may include a data processing component (e.g., an image reconstruction component), a storage component, a display component, etc., that remain still during the medical procedure.

Conventionally, some CT systems achieve the data transmission between the rotating part and the stationary part using WiFi or radio frequency techniques. However, since there is a relative motion between the rotating part and the stationary part during scanning, Doppler effect and multipath effect may exist during the data transmission between the rotating part and the stationary part, which increases the transmission bit error rate and packet loss rate during the data transmission, thereby reducing the efficiency and accuracy of the data transmission. Some CT systems achieve the data transmission between the rotating part and the stationary part through wireless capacitive coupling. Due to the influence of the external electromagnetic environment, the efficiency and accuracy of the data transmission in this way are limited.

In order to improve the data transmission accuracy and efficiency in a medical device, an aspect of the present disclosure provides a data transmission system used in the medical device. The medical device may include a rotating part that rotates during a medical procedure and a stationary part that keeps still during the medical procedure. The data transmission system may include a radio frequency (RF) emitter and an RF receiver. The RF emitter may be mounted on one of the rotating part and the stationary part, and the RF receiver may be mounted on the other one of the rotating part and the stationary part. The RF emitter may be configured to generate a target RF signal encoding target data based on a current relative position of the RF emitter with respect to the RF receiver, and transmit the target RF signal to the RF receiver. The RF receiver may be configured to receive the target RF signal and extract the target data from the target RF signal. The data transmission system in the present disclosure may improve the efficiency and accuracy of the data transmission by taking the relative position of the RF emitter with respect to the RF receiver into consideration and reducing or eliminating impacts of the Doppler effect or the multipath effect.

Another aspect of the present disclosure provides another data transmission system used in the medical device. The data transmission system may include an optical source and at least one photoelectric receiver. The optical source may be mounted on one of the rotating part and the stationary part, and the at least one photoelectric receiver may be mounted on the other one of the rotating part and the stationary part. The optical source may include optical emitters configured to emit optical signals encoding target data. When the rotating part rotates during the medical procedure, at least one of the optical emitters may move into a detection zone of the at least one photoelectric receiver, and the at least one photoelectric receiver may be configured to detect the optical signals from the at least one optical emitter and extract the target data from the detected optical signals. Compared with the conventional CT systems that use wireless capacitive coupling to transmit data, the data transmission system in the present disclosure may be less susceptible to external electromagnetic environment and improve the efficiency of the data transmission by using the optical source and the at least one photoelectric receiver to transmit data.

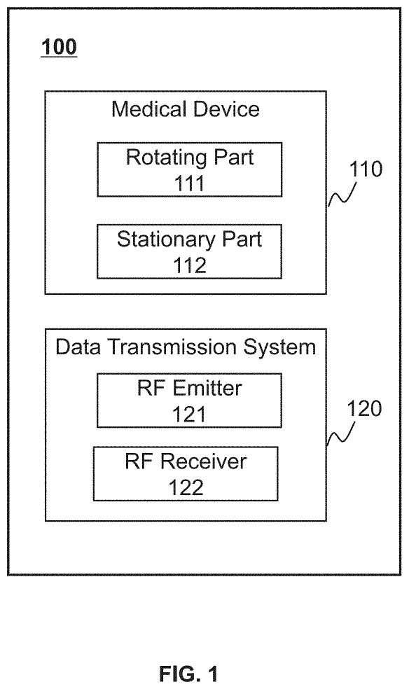

FIG. 1 is a schematic diagram illustrating an exemplary medical system 100 according to some embodiments of the present disclosure.

As shown in FIG. 1, the medical system 100 may include a medical device 110 and a data transmission system 120.

The medical device 110 may be configured to generate or provide image data related to a subject via scanning the subject. In some embodiments, the subject may include a biological subject and/or a non-biological subject. For example, the subject may include a specific portion of a body, such as a heart, a breast, or the like. In some embodiments, the medical system may include a single modality imaging system and/or a multi-modality imaging system. The single modality imaging system may include, for example, an X-ray imaging system, a computed tomography (CT) system, or the like, or any combination thereof. The multi-modality imaging system may include, for example, an X-ray imaging-magnetic resonance imaging (X-ray-MRI) system, a positron emission tomography-X-ray imaging (PET-X-ray) system, a positron emission tomography-computed tomography (PET-CT) system, a C-arm system, etc. It should be noted that the imaging system described below is merely provided for illustration purposes, and not intended to limit the scope of the present disclosure.

In some embodiments, the medical device 110 includes a rotating part 111 and a stationary part 112. The rotating part 111 may rotate during a medical procedure of a subject, and include component(s) that can collect medical data of the subject. The medical procedure may include a medical imaging procedure for obtaining an image of a subject, or a radiotherapy procedure for treating a subject. For illustration purposes, the medical imaging procedure is described hereinafter as an example of the medical procedure.

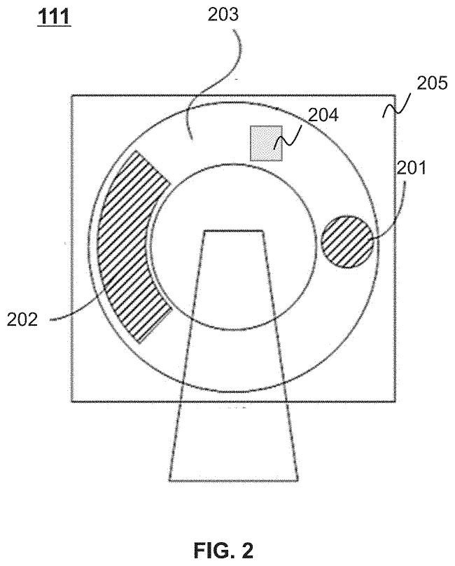

Merely by way of example, FIG. 2 is a schematic diagram illustrating an exemplary rotating part of the medical device 110 according to some embodiments of the present disclosure. As shown in FIG. 2, the rotating part 111 may include a radiation source 201, a detector 202, a rotating gantry 203, etc. The rotating gantry 203 may support the radiation source 201 and the detector 202. During a scan of a subject, the rotating gantry 203 may periodically rotate around the subject located in a scanning area to drive the radiation source 201 and the detector 202 to rotate. In some embodiments, during the scan of the subject, the rotating gantry 203 may have a same motion speed and different motion directions. The radiation source 201 may be configured to generate and/or emit radiation rays (e.g., X-rays) toward the subject, and the detector 202 may be configured to collect scan data by detecting the radiation rays that pass through the subject.

The stationary part 112 may keep still during the medical procedure, and include component(s) that can process the medical data of the subject to generate an image of the subject. For example, the stationary part may include an image reconstruction component, a storage component, one or more terminals, etc. The image reconstruction component may be configured to reconstruct an image of the subject based on the scan data. The storage component may be configured to store data instructions, and/or any other information. In some embodiments, the storage module may store data obtained from one or more components (e.g., the medical device 110, the data transmission system 120, etc.) of the medical system 100. The terminal(s) may be configured to enable a user interaction between a user and the medical system 100. For example, the terminal(s) may receive an instruction to cause the detector 202 to scan the subject. As another example, the terminal(s) may receive a processing result (e.g., the image of the subject) from the image reconstruction component and display the processing result to the user.

In some embodiments, the stationary part 112 may be mechanically connected with the rotating part 111. For example, the component(s) of the stationary part 112 may be integrated in a fixed gantry (e.g., a fixed gantry 205 shown in FIG. 2) outside the rotating part 111. Alternatively or optionally, the component(s) of the stationary part 112 may include individual devices (e.g., an individual computer, an individual console, etc.) that are not mechanically connected to the rotating part 111.

The data transmission system 120 may enable data transmission between the rotating part 111 and the stationary part 112. The data transmitted between the rotating part 111 and the stationary part 112 may include, for example, scan data, a control signal, test data, or the like, or any combination thereof. The data transmission system 120 may include a radio frequency (RF) emitter 121 and an RF receiver 122. The RF emitter 121 may be mounted on one of the rotating part 111 and the stationary part 112, and the RF receiver 122 may be mounted on the other one of the rotating part 111 and the stationary part 112. For example, as shown in FIG. 2, an RF emitter 204 may be mounted on the rotating gantry 203 of the rotating part 111. Accordingly, the RF receiver 122 may be mounted on the stationary part 112. During the scan of the subject, the RF emitter 204 may rotate with the rotating gantry 203.

The RF emitter 121 may be configured to generate a target RF signal encoding target data (e.g., the scan data of the subject). For example, the RF emitter 121 may be mounted on the rotating part 111, the RF receiver 122 may be mounted on the stationary part 112, and the target data may include scan data (e.g., projection data) collected by a detector (e.g., the detector 202) of the medical device 110 during the medical procedure. As another example, the RF emitter 121 may be mount on the stationary part 112, the RF receiver 122 may be mounted on the rotating part 111, the target data may include a control signal for controlling the rotating part 111 to rotate around the subject.

As described elsewhere in the present disclosure, since there is a relative motion between the rotating part 111 and the stationary part 112 during scanning, that is, there is a relative motion between the RF emitter 121 and the RF receiver 122, Doppler effect and/or multipath effect may exist during the data transmission between the RF emitter 121 and the RF receiver 122, which may reduce the efficiency and accuracy of the data transmission. In some embodiments, to reduce or eliminate the Doppler effect and/or the multipath effect, the RF emitter 121 may generate the target RF signal based on a current relative position of the RF emitter 121 with respect to the RF receiver 122, and transmit the target RF signal to the RF receiver 122.

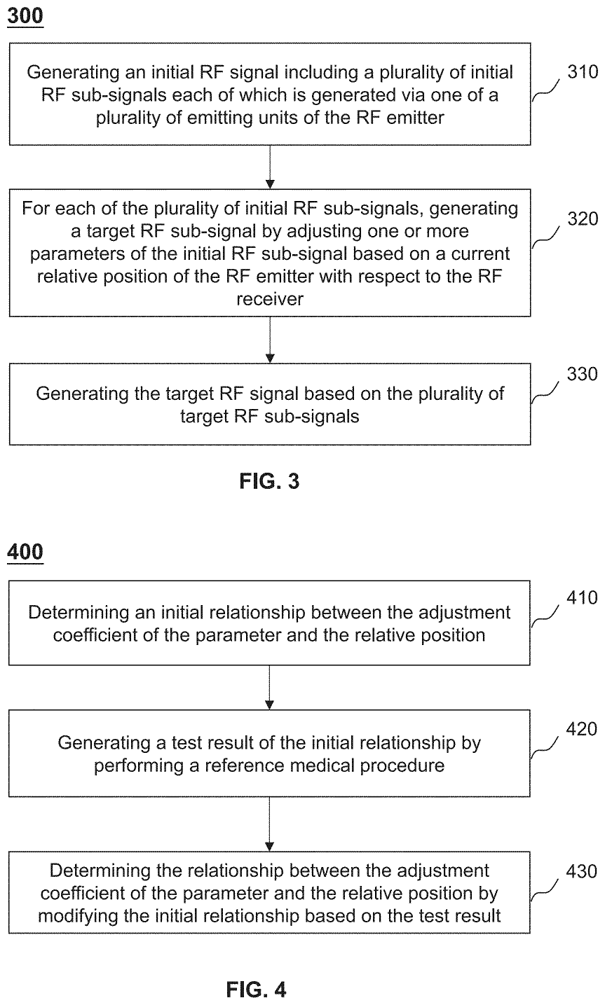

In some embodiments, the RF emitter 121 may include a plurality of emitting units. The RF emitter 121 may generate an initial RF signal including a plurality of initial RF sub-signals each of which is generated via one of the plurality of emitting units of the RF emitter 121. For each of the plurality of initial RF sub-signals, the RF emitter 121 may generate a target RF sub-signal by adjusting one or more parameters of the initial RF sub-signal based on the current relative position of the RF emitter 121 with respect to the RF receiver 122. Further, the RF emitter 121 may generate the target RF signal based on the plurality of target RF sub-signals. More descriptions regarding the generation of the target RF signal may be found elsewhere in the present disclosure (e.g., FIG. 3 and the descriptions thereof).

The RF receiver 122 may be configured to receive the target RF signal and extract the target data from the target RF signal. For example, the RF receiver 122 may extract the target data by decoding the target RF signal. In some embodiments, the target data may include the scan data of the subject collected during the medical procedure. In some embodiments, the RF receiver 122 may be further configured to transmit the scan data extracted from the target RF signal to the image reconstruction component, and the image reconstruction component may reconstruct the image of the subject based on the scan data using an image reconstruction technology.

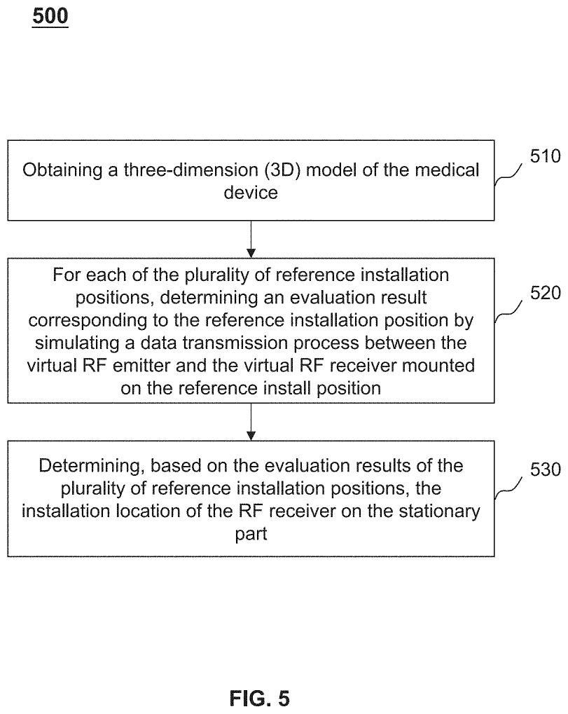

In some embodiments, the RF receiver 122 may be mounted on the stationary part 112, and an installation location of the RF receiver 122 on the stationary part 112 may be determined by performing the following operations. A three-dimension (3D) model of the medical device 110 may be obtained. The 3D model may include a virtual rotating part, a virtual RF emitter mounted on the virtual rotating part, a virtual stationary part, and a plurality of virtual RF receivers mounted on a plurality of reference installation positions of the virtual stationary part. For each of the plurality of reference installation positions, an evaluation result corresponding to the reference installation position may be determined by simulating a data transmission process between the virtual RF emitter and the virtual RF receiver mounted on the reference install position. Further, the installation location of the RF receiver 122 on the stationary part 112 may be determined based on the evaluation results of the plurality of reference installation positions. More descriptions regarding the installation location of the RF receiver 122 on the stationary part 112 may be found elsewhere in the present disclosure (e.g., FIG. 5 and the descriptions thereof).

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. However, those variations and modifications do not depart the scope of the present disclosure. In some embodiments, the medical system 100 may include one or more additional components and/or one or more components described above may be omitted.

For example, the RF emitter 121 may include a processing device configured to generate the target RF signal encoding target data and transmit the target RF signal to the RF receiver 122. As another example, the medical system 100 may include an independent processing device configured to process data and/or information obtained from one or more components (e.g., the medical device 110, the data transmission system 120, etc.) of the medical system 100 to execute the data transmission methods in the present disclosure.

In some embodiments, the medical system 100 in the present disclosure may also include multiple processing devices, and thus operations of a method that are performed by one processing device as described in the present disclosure may also be jointly or separately performed by the multiple processing devices. As another example, the medical system 100 may include a network. The network may include any suitable network that can facilitate the exchange of information and/or data for the medical system 100. In some embodiments, one or more components of the medical system 100 may communicate information and/or data with one or more other components of the medical system 100 via the network.

FIG. 3 is a flowchart illustrating an exemplary process 300 for generating a target RF signal according to some embodiments of the present disclosure. In some embodiments, process 300 may be executed by the medical system 100. For example, the process 300 may be implemented as a set of instructions (e.g., an application) stored in a storage device. In some embodiments, a processing device (e.g., the processing device of the RF emitter 121, an independent processing device) may execute the set of instructions and may accordingly be directed to perform the process 300.

In 310, the RF emitter 121 may generate an initial RF signal including a plurality of initial RF sub-signals each of which is generated via one of a plurality of emitting units of the RF emitter 121.

In some embodiments, an initial RF sub-signal may be generated by performing an encoding operation on a portion of the target data by one of the plurality of emitting units of the RF emitter 121. Exemplary encoding operations may include data format normalization, data compression, or the like, or any combination thereof.

In 320, for each of the plurality of initial RF sub-signals, the RF emitter 121 may generate a target RF sub-signal by adjusting one or more parameters of the initial RF sub-signal based on a current relative position of the RF emitter 121 with respect to the RF receiver 122.

In some embodiments, a relative position of the RF emitter 121 with respect to the RF receiver 122 may be represented by a rotation angle of the rotating part 111 (e.g., a rotation angle of the rotating gantry 203). In some embodiments, the relative position of the RF emitter 121 with respect to the RF receiver 122 may be represented by a difference between coordinates of the RF emitter 121 and the RF receiver 122 in a coordinate system.

In some embodiments, a current relative motion speed and a current relative motion direction of the RF emitter 121 with respect to the RF receiver 122 may be acquired via one or more position measurement devices. Exemplary position measurement devices may include a magnetic grid ruler, an encoder, an accelerometer, or the like, or any combination thereof. The one or more position measurement devices may communicate with the RF emitter 121 via any suitable network (e.g., a Bluetooth™ network, a ZigBee™ network, a Wi-Fi network, a wired network, etc.) that can facilitate the exchange of information and/or data. Further, the current relative position of the RF emitter 121 with respect to the RF receiver 122 may be determined based on the current relative motion speed and the current relative motion direction of the RF emitter 121 with respect to the RF receiver 122.

In some embodiments, the one or more parameters of an initial RF sub-signal include a carrier frequency, an encoding mode, a modulation mode, a magnitude, a phase, or the like, or any combination thereof. The encoding mode and the modulation mode of an initial RF sub-signal may affect the transmission speed of the initial RF sub-signal.

In some embodiments, for each of the one or more parameters of the initial RF sub-signal, the RF emitter 121 may obtain a relationship between an adjustment value of the parameter and a relative position of the RF emitter 121 with respect to the RF receiver 122. In some embodiments, the relationship between the adjustment value of the parameter and the relative position may be a relationship between the adjustment value of the parameter and a relative position range. For example, if there are 72 relative position ranges corresponding to rotation angle ranges (1°-5°), (6°-10°), . . . , (351°-355°), and (355°-360°) of the rotating part 111 during a rotation period of the rotating part 111, the relationship between the adjustment value of the parameter and the relative position may be a corresponding relationship between the 72 relative position ranges and the adjustment values of the parameter corresponding to the 72 relative position ranges.

In some embodiments, since the carrier frequency is affected by the Doppler effect during the data transmission, the adjustment value of the carrier frequency refers to a value by which the carrier frequency needs to be adjusted to compensate for an impact of the Doppler effect corresponding to the current relative position. In some embodiments, since the encoding mode and/or the modulation mode may be affected by the multipath effect, an adjustment value of the encoding mode and/or the modulation mode refers to a value by which the encoding mode and/or the modulation mode needs to be adjusted to compensate for an impact of the multipath effect corresponding to the current relative position. In some embodiments, the relationship between the adjustment value of the encoding mode and/or the modulation mode and the relative position may include relationships between the adjustment value of the encoding mode and/or the modulation mode and the relative position corresponding to different carrier frequency bands for transmitting RF signal. In some embodiments, in order to enable the RF receiver 122 to receive desired signal, it is necessary to perform beamforming on each initial RF sub-signal, that is, the magnitude and the phase of each initial RF sub-signal need to be adjusted. The adjustment values of the magnitude and the phase refers to values by which the magnitude and the phase needs to be adjusted when the beamforming is performed on the initial RF sub-signal.

In some embodiments, the relationship between the adjustment value of the parameter and the relative position may indicate the adjustment value of the parameter of each initial RF sub-signal corresponding to each of a plurality of possible relative positions. The adjustment values of the parameter of different initial RF sub-signals may be the same or different. Merely by way of example, the RF emitter 121 includes N emitting units, and there are 72 possible relative positions. The relationship corresponding to the phase may indicate the adjustment values of the phase of N initial RF sub-signals emitted by the N emitting units when the RF emitter 121 is located at each possible relative position with respect to the RF receiver 122. As another example, the adjustment values of the phase of different initial RF sub-signals may be the same, and the relationship only needs to indicate the adjustment value of the phase of one initial RF sub-signal when the RF emitter 121 is located at each possible relative position with respect to the RF receiver 122.

In some embodiments, the RF emitter 121 may obtain the relationship between the adjustment value of a parameter and the relative position from one or more components of the medical system 100 (e.g., the storage device, the terminals(s)) or an external source via a network. For example, the relationship between the adjustment value of the parameter and the relative position may be previously generated by a computing device, and stored in a storage device. The RF emitter 121 may access the storage device and retrieve the relationship between the adjustment value of the parameter and the relative position. More descriptions regarding the generation of the relationship between the adjustment value of the parameter and the relative position may be found elsewhere in the present disclosure (e.g., FIG. 4 and the descriptions thereof).

Further, for each of the one or more parameters of the initial RF sub-signal, the RF emitter 121 may adjust the parameter based on the relationship and the current relative position of the RF emitter with respect to the RF receiver. In some embodiments, the relationship may be represented as a look-up table. The look-up table may include multiple relative positions and their corresponding adjustment values. For example, the look-up table may include multiple relative position ranges and their corresponding adjustment values. The RF emitter 121 may identify the adjustment value of the parameter corresponding to the current relative position from the look-up table according to the current relative position. For example, if the look-up table includes multiple relative positions and their corresponding adjustment values, the RF emitter 121 may determine a closest relative position to the current relative position from multiple relative positions, and designate the adjustment value of the parameter corresponding to the closest relative position as the adjustment value of the parameter corresponding to the current relative position. As another example, if the look-up table includes multiple relative position ranges and their corresponding adjustment values, the RF emitter 121 may determine a relative position range where the current relative position is located in, and designate the adjustment value of the parameter corresponding to the relative position range as the adjustment value of the parameter corresponding to the current relative position.

Then, the RF emitter 121 may adjust the parameter according to the adjustment value of the parameter corresponding to the current relative position.

For example, for the carrier frequency of the initial RF sub-signal, the RF emitter 121 may directly identify the adjustment value of the carrier frequency corresponding to the current relative position from the look-up table according to the current relative position (or the relative position range corresponding to the current relative position), and further adjust the carrier frequency according to the adjustment value of the carrier frequency corresponding to the current relative position. As another example, for the encoding mode and/or the modulation mode of the initial RF sub-signal, the RF emitter 121 may first determine the frequency band for transmitting the initial RF sub-signal. Then, the RF emitter 121 may identify the adjustment value of the encoding mode and/or the modulation mode corresponding to the current relative position from the look-up table according to the current relative position (or the relative position range corresponding to the current relative position) and the frequency band. Further, the RF emitter 121 may adjust the encoding mode and/or the modulation mode according to the adjustment value of the encoding mode and/or the modulation mode corresponding to the current relative position. As still another example, the RF emitter 121 may perform the beamforming on the initial RF sub-signal to adjust the directivity of the initial RF sub-signal transmission (i.e., pointing to the RF receiver 122), thereby optimizing the transmission of the initial RF sub-signal and improving the efficiency of the transmission of the initial RF sub-signal. Specifically, the RF emitter 121 may identify the adjustment values of the magnitude and the phase corresponding to the current relative position from the look-up table according to the current relative position (or the relative position range corresponding to the current relative position), and further adjust the magnitude and the phase according to the adjustment value of the magnitude and the phase corresponding to the current relative position.

Then, the initial RF sub-signal obtained after the one or more parameters of the initial RF sub-signal is adjusted may be designated as the target RF sub-signal corresponding to the initial RF sub-signal.

In 330, the RF emitter 121 may generate the target RF signal based on the plurality of target RF sub-signals.

For example, the RF emitter 121 may combine the plurality of target RF sub-signals into the target RF signal.

According to some embodiments, a target RF sub-signal may be generated by adjusting the frequency and/or the transmission speed of each initial RF sub-signal based on the current relative position of the RF emitter 121 with respect to the RF receiver 122, which may reduce or eliminate impacts of the Doppler effect and/or the multipath effect on the initial RF signal, thereby improving the efficiency and accuracy of the data transmission. Moreover, in some embodiments, the magnitude and the phase of each initial RF sub-signal may be also adjusted to improve the directivity of the target RF signal toward the RF receiver 122, thereby further improving the efficiency and accuracy of the data transmission.

It should be noted that the above description regarding the process 300 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the RF emitter 121 may include only one emitting unit configured to generate an initial RF signal, the RF emitter 121 may generate a target RF signal by adjusting one or more parameters of the initial RF signal in a similar manner as that described in process 300.

FIG. 4 is a flowchart illustrating an exemplary process 400 for determining a relationship between an adjustment value of a parameter of an RF signal and a relative position of the RF emitter 121 with respect to the RF receiver 122 according to some embodiments of the present disclosure. In some embodiments, process 400 may be executed by the medical system 100. For example, the process 400 may be implemented as a set of instructions (e.g., an application) stored in a storage device. In some embodiments, a processing device (e.g., the processing device of the RF emitter 121, an independent processing device) may execute the set of instructions and may accordingly be directed to perform the process 400.

The RF signal described in FIG. 4 refers to an RF signal generated via an emit unit of an RF emitter. For example, the RF signal may be an initial RF sub-signal generated via an emit unit of the RF emitter 121 described in FIG. 3.

In 410, an initial relationship between the adjustment value of the parameter and the relative position may be determined.

As described elsewhere in the present disclosure, since the rotating gantry 203 may periodically rotate, the relative motion between the RF emitter 121 and the RF receiver 122 may be periodic. Thus, the relationship between the adjustment value of the parameter and the relative position may be represented via relative positions during a rotation period of the rotating part 111 and corresponding adjustment value of each relative position.

In some embodiments, possible relative positions of the RF emitter 121 with respect to the RF receiver 122 during a rotation period of the rotating part 111 (e.g., a rotation period of the rotating gantry 203) may be determined. In some embodiments, the possible relative positions may be set manually by a user (e.g., an engineer) according to experience or automatically by the medical system 100. Merely by way of example, the possible relative positions may be set according to a preset rotation step of the rotating part 111. For example, if the preset rotation step of the rotating part 111 is 5°, there are 72 possible relative positions corresponding to rotating angles 5°, 10°, . . . , 355°, 360° of the rotating part 111 in the rotation period.

Then, for each of the possible relative positions, an initial adjustment value of the parameter corresponding to the possible relative position may be determined. For example, for each of the possible relative positions, the initial adjustment value of the parameter corresponding to the possible relative position may be determined by simulating a data transmission process between the RF emitter 121 and the RF receiver 122 when the RF emitter 121 and the RF receiver 122 are at the possible relative position. Further, the initial relationship between the adjustment value of the parameter and the relative position may be determined according to the initial adjustment values of the parameter and the possible relative positions. For example, the initial adjustment values of the parameter and the possible relative positions may be filled into an initial look-up table according to their corresponding relationship to obtain the initial relationship. As another example, for each of the possible relative positions, an initial relative position range including the possible relative position may be determined, and the initial adjustment value of the parameter corresponding to the possible relative position may be designated as an initial adjustment value of the parameter corresponding to the initial relative position range. For example, the initial relative position range including the possible relative position may be a relative position range between the possible relative position and its adjacent relative possible position.

In some embodiments, for a possible relative position, an impact of at least one of the Doppler effect or the multipath effect corresponding to the possible relative position may be determined, and an initial adjustment value of the parameter corresponding to the possible relative position may be determined based on the estimated impact. As used herein, the impact of at least one of the Doppler effect or the multipath effect corresponding to the possible relative position refers to the impact of at least one of the Doppler effect or the multipath effect on the parameter when the RF emitter 121 is located at the possible relative position with respect to the RF receiver 122.

For example, if the parameter is the carrier frequency, for each of the possible relative positions, an impact of the Doppler effect on the carrier frequency when the RF emitter 121 and the RF receiver 122 are at the possible relative position may be estimated, and an initial adjustment value of the carrier frequency corresponding to the possible relative position may be determined based on the estimated impact of the Doppler effect. The initial adjustment value of the carrier frequency may be used to compensate the estimated impact of the Doppler effect on the carrier frequency. Merely by way of example, when the RF emitter 121 moves to the RF receiver 122, due to the Doppler effect, the carrier frequency of the RF signal received by RF receiver 122 may be higher than the carrier frequency of the RF signal emitted by the RF emitter 121. At this case, the initial adjustment value corresponding to the carrier frequency may be negative number. As another example, when the RF emitter 121 moves away from the RF receiver 122, due to the Doppler effect, the carrier frequency of the RF signal received by RF receiver 122 may be lower than the carrier frequency of the RF signal emitted by the RF emitter 121. At this case, the initial adjustment value corresponding to the carrier frequency may be positive number.

As another example, if the parameter is the encoding mode and/or the modulation mode, for each of the possible relative positions, an impact of the multipath effect on the encoding mode and/or the modulation mode when the RF emitter 121 and the RF receiver 122 are at the possible relative position may be estimated, and an initial adjustment value of the encoding mode and/or the modulation mode corresponding to the possible relative position may be determined based on the estimated impact of the multipath effect. Multipath effect may cause that the RF signal received by RF receiver 122 has some changes, such as a decline in the signal-to-noise ratio and a phase shift. The initial adjustment value of the encoding mode and/or the modulation mode may be used to compensate the estimated impact of the multipath effect. In some embodiments, the impacts of the multipath effect are different in different carrier frequency bands for transmitting RF signals. The determined initial adjustment value of the encoding mode and/or the modulation mode may correspond to the possible relative position and the carrier frequency band for transmitting the RF signal emitted by the RF emitter 121.

In some embodiments, if the parameter is the magnitude and the phase, for each of the possible relative positions, a beam direction and a beam width of the RF signal may be determined according to the possible relative position, and then initial adjustment values of the magnitude and the phase corresponding to the possible relative position may be determined according to the beam direction and the beam width. The initial adjustment values of the magnitude and the phase may be used to perform a beamforming on the RF signal emitted by the RF emitter 121 so that the RF signal received by the RF receiver 122 may satisfy needs.

In some embodiments, for each two adjacent possible relative positions, it may be determined whether a difference between the initial adjustment values of the parameter corresponding to the two adjacent possible relative positions is greater than a first difference threshold; in response to determining that the difference is greater than the first difference threshold, one or more additional possible relative positions between the two adjacent possible relative positions may be added, and the initial adjustment values corresponding to the one or more additional possible relative positions may be determined. In some embodiments, for each two adjacent possible relative positions, it may be determined whether a difference between the initial adjustment values of the parameter corresponding to the two adjacent possible relative positions is smaller than a second difference threshold; in response to determining that the difference is smaller than the second difference threshold, one of the two possible relative positions may be deleted.

In some embodiments, the initial relationship may be directly designated as the relationship between the adjustment value of the parameter and the relative position. Alternatively or optionally, the relationship between the adjustment value of the parameter and the relative position may be determined by performing operations 420 and 430.

In 420, a test result of the initial relationship may be generated by performing a reference medical procedure.

In some embodiments, the reference medical procedure may be performed by the medical device. During the reference medical procedure, the rotating part 111 may rotate one or more rotation periods, when the rotating part 111 rotates to a specific position, the RF emitter 121 may generate a reference RF signal based on the initial relationship and a specific relative position of the RF emitter 121 with respect to the RF receiver 122, and transmit the reference RF signal to the RF receiver 122. The generation of the reference RF signal may be performed in a similar manner as the generation of the target RF signal based on the current relative position and the relationship as described in connection with FIG. 3. A quality of the reference RF signal received by the RF receiver 122 may be evaluated to obtain a test result corresponding to the specific relative position. In some embodiments, the quality of the reference RF signal may be evaluated by analyzing one or more quality parameters of the reference RF signal. Exemplary quality parameters may include a signal-to-noise ratio, a bit error rate, a packet loss rate, etc., or the like, or any combination thereof.

In some embodiments, the quality of the reference RF signal may be evaluated by comparing one or more quality parameters of the reference RF signal to one or more parameters of reference RF signals corresponding to other relative positions. For example, if the signal-to-noise ratio of the reference RF signal corresponding to the specific relative position is substantially the same as the signal-to-noise ratios of the reference RF signals corresponding to other relative positions, the quality of the reference RF signal may be deemed as satisfying requirements. As used herein, substantially, when used to describe a feature (e.g., the feature of A being the same as B), indicates that the deviation from the feature is below a threshold. Merely by way of example, two signal-to-noise ratios are substantially the same may indicate that a difference between the two signal-to-noise ratios is smaller than a threshold. Therefore, in some embodiments, the quality of the reference RF signal may be compared with qualities of reference RF signals corresponding to other specific relative position adjacent to the specific relative position to obtain the test result corresponding to the specific relative position.

In some embodiments, the rotating part 111 may include a non-idle state and an idle state. As used herein, in the idle state, the medical device is not performing a scan and the rotating part 111 is still. In non-idle state, the medical device is performing a scan and the rotating part 111 is rotating. Before the reference medical procedure is performed, it may be determined whether the rotating part 111 is in the non-idle state or the idle state. In response to determining that the rotating part 111 is in the idle state, the reference medical procedure may be performed.

In 430, the relationship between the adjustment value of the parameter and the relative position may be determined by modifying the initial relationship based on the test result.

For a specific relative position, whether the initial adjustment value corresponding to the specific relative position needs to modified may be determined based on the test result corresponding to the specific relative position. For example, for the specific relative position, if the corresponding test result indicates that the quality of the reference RF signal does not satisfy requirements, the initial adjustment value corresponding to the specific relative position may be modified. Merely by way of example, if the quality of the reference RF signal does not satisfy requirements because a carrier frequency of the reference RF signal is smaller than a desire carrier frequency, the initial adjustment value of the carrier frequency corresponding to the specific relative position may be increased. As another example, if the quality of the reference RF signal does not satisfy requirements because the one or more quality parameters of the reference RF signal and one or more quality parameters of reference RF signals corresponding to other specific relative positions are not substantially the same, the initial adjustment value corresponding to the specific relative position may be modified according to the one or more quality parameters of reference RF signals corresponding to other specific relative position. If the corresponding test result indicates that the quality of the reference RF signal satisfies requirements, the initial adjustment value corresponding to the reference relative position may not be modified. The modified initial relationship may be designated as the relationship between the adjustment value of the parameter and the relative position.

According to the process 400, the obtained initial relationship between the adjustment value of the parameter and the relative position may have a relatively high accuracy. In some embodiments, the initial relationship may be further adjusted based on the test result of the initial relationship to obtain the relationship between the adjustment value of the parameter and the relative position, which may further improve the accuracy of the relationship.

FIG. 5 is a flowchart illustrating an exemplary process 500 for determining an installation location of the RF receiver 122 on the stationary part 112 according to some embodiments of the present disclosure. In some embodiments, process 500 may be executed by the medical system 100. For example, the process 500 may be implemented as a set of instructions (e.g., an application) stored in a storage device. In some embodiments, a processing device (e.g., the processing device of the RF emitter 121, an independent processing device) may execute the set of instructions and may accordingly be directed to perform the process 500.

As described elsewhere in the present disclosure, the RF emitter 121 may be mounted on the rotating part 111, and the RF receiver 122 may be mounted on the stationary part 112. The installation location of the RF receiver 122 on the stationary part 112 may be determined by performing the following operations.

In 510, a three-dimension (3D) model of the medical device 110 may be obtained.

In some embodiments, the 3D model may include a virtual rotating part representing the rotating part 111, a virtual RF emitter representing the RF emitter 121 mounted on the virtual rotating part, a virtual stationary part representing the stationary part 112, and a plurality of virtual RF receivers representing the RF receiver 122 mounted on a plurality of reference installation positions of the virtual stationary part. The virtual RF emitter may be mounted on the virtual rotating part, and the plurality of virtual RF receivers may be mounted on a plurality of reference installation positions of the virtual stationary part.

In some embodiments, the 3D model may be previously generated by a computing device, and stored in a storage device. The 3D model may be obtained by accessing the storage device.

In 520, for each of the plurality of reference installation positions, an evaluation result corresponding to the reference installation position may be determined by simulating a data transmission process between the virtual RF emitter and the virtual RF receiver mounted on the reference install position.

For example, in a simulated data transmission process corresponding to a reference install position, the virtual RF emitter may emit a simulated RF signal toward the virtual RF receiver mounted on the reference install position, and the virtual RF receiver 122 may receive the simulated RF signal. The evaluation result corresponding to the reference installation position may be determined by analyzing one or more parameters of the simulated RF signal received by the virtual RF signal mounted on the reference installation position. Exemplary parameters may include a signal-to-noise ratio, a bit error rate, a packet loss rate, a transmission speed of the virtual RF signal received by the virtual RF receiver, a difference between the virtual RF signal emitted by the virtual RF emitter and the virtual RF signal received by the virtual RF receiver, or the like, or any combination thereof. For example, the evaluation result corresponding to the reference installation position may be represented by a score. If the signal-to-noise ratio of the virtual RF signal received by the virtual RF receiver is relatively high, the evaluation result corresponding to the reference installation position may have a relatively large score. If the bit error rate is relatively small, the evaluation result corresponding to the reference installation position may have a relatively large score. If the packet loss rate is relatively small, the evaluation result corresponding to the reference installation position may have a relatively large score. If the difference between the virtual RF signal emitted by the virtual RF emitter and the virtual RF signal received by the virtual RF receiver is relatively small, the evaluation result corresponding to the reference installation position may have a relatively large score.

In 530, the installation location of the RF receiver 122 on the stationary part 112 may be determined based on the evaluation results of the plurality of reference installation positions.

In some embodiments, a reference installation position with a maximum score may be designated as the installation location of the RF receiver 122 on the stationary part 112. In some embodiments, one or more reference installation positions whose scores are greater than a score threshold may be determined from the plurality of reference installation positions, and the one or more reference installation positions may be determined as one or more target installation positions. Any one of the one or more target installation positions may be designated as the installation location of the RF receiver 122 on the stationary part 112. For example, a user may manually select one target installation position as the installation location of the RF receiver 122.

According to the process 500, the evaluation result corresponding to the reference installation position may be determined by simulating the data transmission process between the virtual RF emitter and the virtual RF receiver mounted on the reference install position instead of performing actual data transmission process. This may improve the efficiency of evaluating the reference install positions, thereby improving the efficiency of the determination of the installation location of the RF receiver 122 on the stationary part 112 based on the evaluation results of the reference installation positions.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof. For example, the operations of the illustrated processes 300, 400, and 500 are intended to be illustrative. In some embodiments, the processes 300, 400, and 500 may be accomplished with one or more additional operations not described, and/or without one or more of the operations discussed. Additionally, the order in which the operations of the processes 300, 400, and 500 and regarding descriptions are not intended to be limiting.

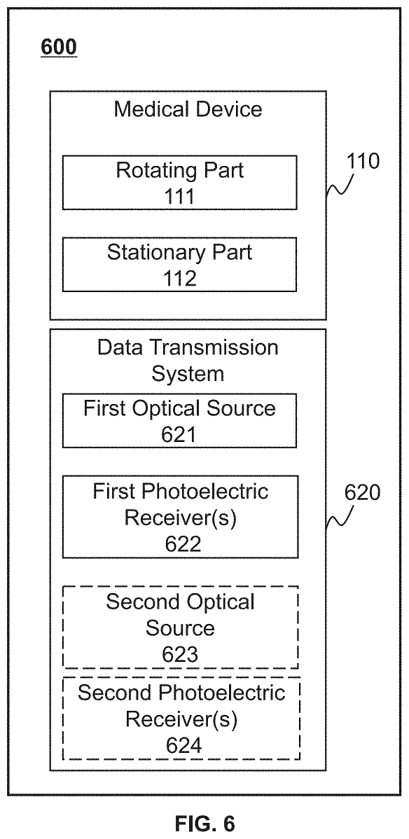

FIG. 6 is a schematic diagram illustrating an exemplary medical system 600 according to some embodiments of the present disclosure.

As shown in FIG. 6, the medical system 600 may be similar to the medical system 100 as described in connection with FIG. 1, except that the data transmission system 120 is replaced by a data transmission system 620.

The data transmission system 620 may enable data transmission between the rotating part 111 and the stationary part 112. The data transmission system 620 may include an optical source 621 (also referred to as a first optical source 621) and one or more photoelectric receivers 622 (also referred to as one or more first photoelectric receivers 622).

In some embodiment, the first optical source 621 may be an optical array including optical emitters configured to emit optical signals encoding target data (e.g., scan data of a subject). In some embodiments, the target data may include raw data collected by a detector of the medical device 110. An optical emitter may be a light source that is capable of encoding data by converting an electrical signal into an optical signal. For example, an optical emitter may include an ultraviolet light source, an infrared light source, a visible light source (e.g., a light emitting diode (LED)), a laser diode (LD), etc., that is capable of encoding data.

The first photoelectric receiver(s) 622 may be configured to detect the optical signals from the first optical source 621 and extract the target data from the detected optical signals. For example, the first photoelectric receiver(s) 622 may extract the target data by decoding the detected optical signals. In some embodiments, the target data may include the scan data of the subject collected during the medical procedure. Exemplary photoelectric receivers may include a positive intrinsic-negative (PIN) diode, an avalanche photo diode (APD), a single photon detector, or the like, or any combination thereof.

The one or more photoelectric receivers may be further configured to transmit the scan data extracted from the detected optical signals to an image reconstruction component of the medical device 110 for reconstructing an image of the subject based on the scan data.

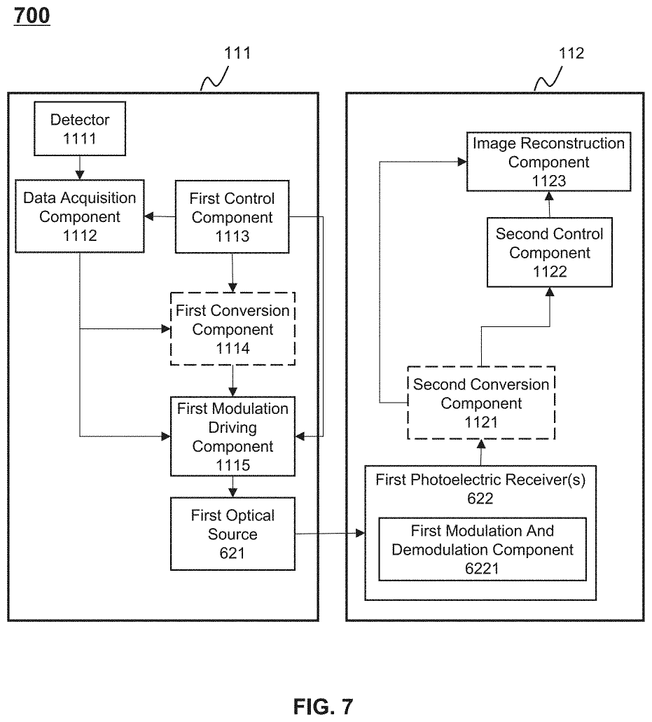

In some embodiments, the first optical source 621 may be mounted on one of the rotating part 111 and the stationary part 112, and the first photoelectric receiver(s) 622 may be mounted on the other one of the rotating part 111 and the stationary part 112. For example, the first optical source 621 may be mounted on the rotating part 111, and the first photoelectric receiver(s) 622 may be mounted on the stationary part 112, which may achieve data transmission from the rotating part 111 to the stationary part 112 (e.g., the transmission of the scan data). More descriptions regarding the data transmission from the rotating part 111 to the stationary part 112 may be found elsewhere in the present disclosure (e.g., FIG. 7 and the descriptions thereof).

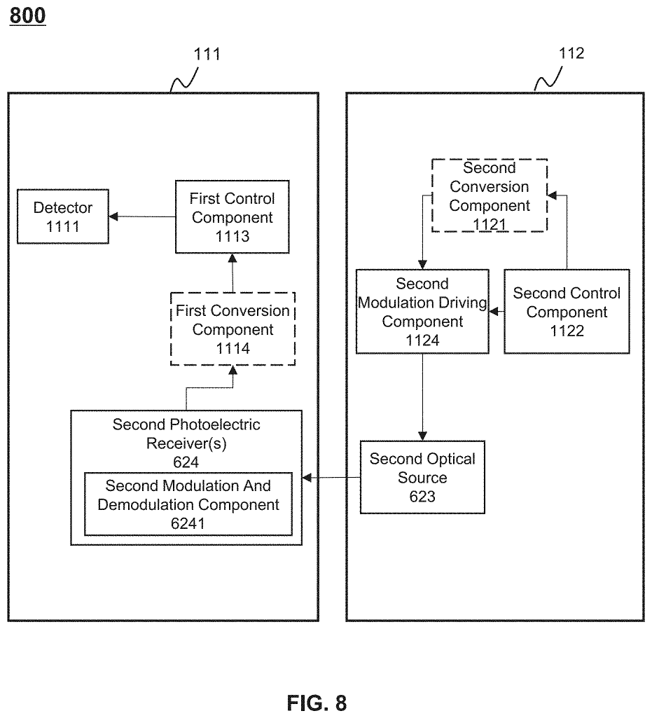

As another example, the first optical source 621 may be mounted on the stationary part 112, and the first photoelectric receiver(s) 622 may be mounted on the rotating part 111, which may achieve data transmission from the stationary part 112 to the rotating part 111 (e.g., the transmission of a control instruction that is used to cause one or more components of the rotating part 111 to perform one or more operations). More descriptions regarding the data transmission from the stationary part 112 to the rotating part 111 may be found elsewhere in the present disclosure (e.g., FIG. 8 and the descriptions thereof).

When the rotating part 111 rotates during the medical procedure, at least one of the optical emitters of the first optical source 621 may move into a detection zone of the photoelectric receiver(s), and the photoelectric receiver(s) may detect the optical signals from the at least one optical emitter and extract the target data from the detected optical signals.

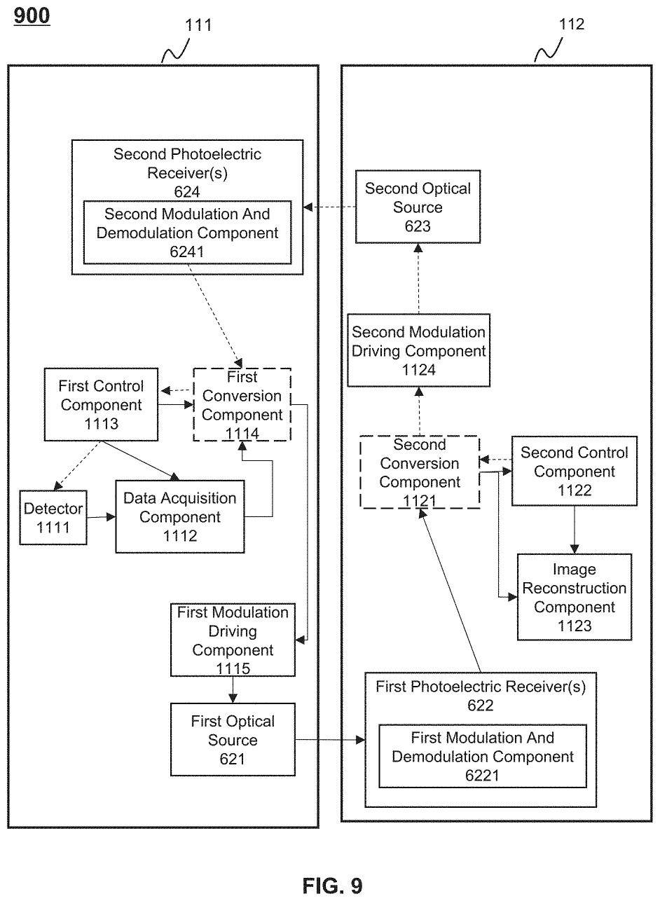

In some embodiments, the data transmission system 620 may include a second optical source 623 and one or more second photoelectric receivers 624. The second optical source 623 may be mounted on the other one of the rotating part 111 and the stationary part 112 where the first photoelectric receiver(s) 622 are mounted on, and the second photoelectric receiver(s) 624 may be mounted on the one of the rotating part 111 and the stationary part 112 where the first optical source 621 is mounted on. For example, if the first optical source 621 is mounted on the rotating part 111 and the first photoelectric receiver(s) 622 are mounted on the stationary part 112, the second optical source 623 may be mounted on the stationary part 112 and the second photoelectric receiver(s) 624 may be mounted on the rotating part 111. As another example, if the first optical source 621 is mounted on the stationary part 112 and the first photoelectric receiver(s) 622 are mounted on the rotating part 111, the second optical source 623 may be mounted on the rotating part 111 and the second photoelectric receiver(s) 624 may be mounted on the stationary part 112. In this way, two-way data transmission may be achieved between the rotating part 111 and the stationary part 112. More descriptions regarding the two-way data transmission may be found elsewhere in the present disclosure (e.g., FIG. 9 and the descriptions thereof).

In some embodiments, one or more components (the first optical source 621, the one or more first photoelectric receivers 622, the second optical source 623, or the one or more second photoelectric receivers 624) of the data transmission system 620 may be arranged inside light shields to avoid interference of other light sources on the data transmission system 620.

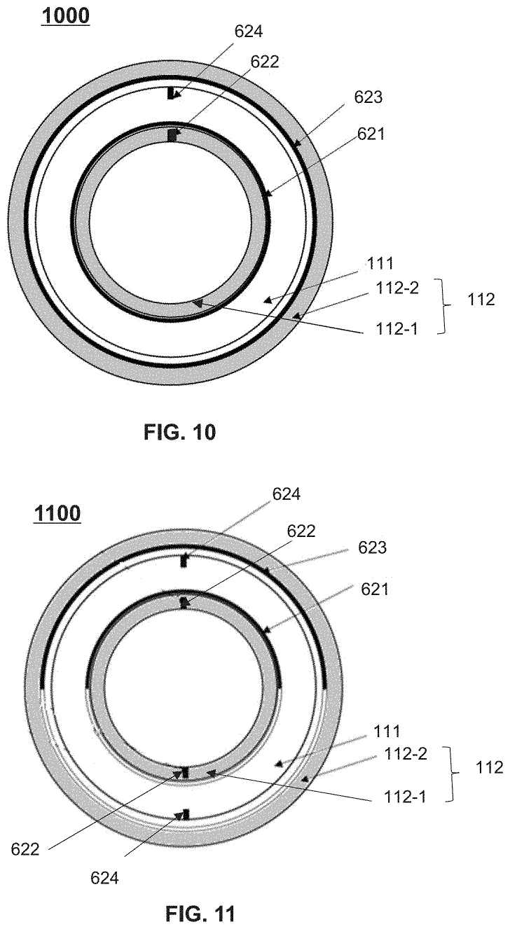

In some embodiments, the data transmission system 620 may transmit the target data in different data transmission modes. For example, the data transmission modes may include a single-input and single-output (SISO) mode, a single-input and multiple-output (SIMO) mode, a multiple-input and single-output (MISO) mode, a multiple-input and multiple-output (MIMO) mode, etc. Merely by way of example, in the SISO mode, the first optical source 621 may cover the rotating part 111 along a circumferential of the rotating part 111, and the first photoelectric receiver(s) 622 may include one photoelectric receiver 622. When the rotating part 111 rotates during the medical procedure, at least one optical emitter of the optical emitters of the first optical source 621 may move into a detection zone of the first photoelectric receiver 622, and the first photoelectric receiver 622 may detect the optical signals from the at least one optical emitter. As another example, in the SIMO mode, the first optical source 621 may cover a portion of the rotating part 111 along a circumferential of the rotating part 111, and the first photoelectric receiver(s) 622 may include a plurality of first photoelectric receivers 622. When the rotating part 111 rotates during the medical procedure, the plurality of first photoelectric receivers 622 sequentially detect the optical signals from the first optical source 621. As still another example, in the MISO mode, the first photoelectric receiver(s) 622 may include one first photoelectric receiver 622. The first optical source 621 may cover the rotating part 111 along the circumferential of the rotating part 111 and include a plurality of optical sub-arrays. When the rotating part rotates during the medical procedure, the plurality of optical sub-arrays sequentially may emit the optical signals to the photoelectric receiver 622. As still another example, in the MIMO mode, the target data may be divided into a plurality sets of data. The first optical source 621 may include a plurality of optical sub-arrays each of which is configured to transmit one set of the plurality sets of data, and the first photoelectric receiver(s) 622 may include a plurality of first photoelectric receivers 622. When the rotating part 111 rotates during the medical procedure, the optical signals from each of the plurality of optical sub-arrays may be detected by one of the plurality of first photoelectric receivers 622. More descriptions regarding the data transmission system for transmitting the scan data of the subject may be found elsewhere in the present disclosure (e.g., FIGS. 10-13 and the descriptions thereof).

FIG. 7 is a schematic diagram illustrating an exemplary medical system 700 according to some embodiments of the present disclosure.

The data transmission system of the medical system 700 may be configured to transmit data from the rotating part 111 to the stationary part 112. For illustration purposes, the implementation of the transmission of scan data of the subject is described hereinafter.

In some embodiments, the rotating part 111 may include a radiation source (not shown), a detector 1111, a gantry (not shown), a data acquisition component 1112, a first control component 1113, a first conversion component 1114, a first modulation driving component 1115, etc. The stationary component 112 may include a second conversion component 1121, a second control component 1122, an image reconstruction component 1123, etc. The data transmission system may include the first optical source 621 and the first photoelectric receiver(s) 622. The first optical source 621 may be mounted on the rotating part 111, and the first photoelectric receiver(s) 622 may be mounted on the stationary part 112.

The detector 1111 may collect the scan data of the subject during the scan of the subject. The first control component 1113 may control the data acquisition component 1112 to obtain the scan data collected by the detector 1111, and send the scan data to the first modulation driving component 1115. In some embodiments, the data acquisition component 1112 may be integrated in the detector 1111. In some embodiments, the first control component 1113 may generate and send control data for controlling operations of one or more components of the medical device (e.g., an instruction for image reconstruction) to the first modulation driving component 1115. In some embodiments, the scan data and the control data may have different formats and need to be transmitted simultaneously through a same transmission channel. Therefore, the first control component 1113 may control the data acquisition component 1112 to send the scan data to the first conversion component 1114, and the first control component 1113 may also send the control data to the first conversion component 1114. The first conversion component 1114 may perform a first format conversion on the scan data and/or the control data to unify the scan data and the control data into the same format for subsequent transmission. For example, the scan data and/or control data may be encoded by using a preset encoding algorithm. Then, the first conversion component 1114 may send the scan data and control data with the same format to the first modulation driving component 1115.

The first modulation driving component 1115 may be configured to drive the first optical source 621 to emit optical signals encoding the scan data and/or the control data. In some embodiments, before the first modulation driving component 1115 drives the first optical source 621 to emit the optical signals, the first modulation driving component 1115 may modulate the scan data and/or the control data. For example, the first modulation driving component 1115 may modulate the scan data and/or the control data to control intensities of the optical signals emitted by the first optical source 621. Exemplary modulation methods may include an on-off keying (OOK), an amplitude shift keying (ASK), a frequency shift keying (FSK), a phase shift keying (PSK), a pulse amplitude modulation (PAM), an orthogonal frequency division multiplexing (OFDM), or the like, or any combination thereof.

The first photoelectric receiver(s) 622 may detect the optical signals from the first optical source 621 and extract the scan data and/or the control data from the detected optical signals, and send the scan data and/or the control data to the second conversion component 1121 or the second control component 1122. In some embodiments, the first photoelectric receiver(s) 622 may include a first modulation and demodulation component 6221 configured to modulate and demodulate the detected optical signals to extract the scan data and control data. In some embodiments, each of the first photoelectric receiver(s) 622 may include a modulation and demodulation sub-component configured to modulate and demodulate the optical signals detected by the first photoelectric receiver 622. Exemplary modulation and demodulation operations may include a signal filtering operation, a signal amplifying operation, an analog-to-digital conversion operation, or the like, or any combination thereof. In some embodiments, the second conversion component 1121 may perform a second format conversion on the scan data and/or the control data to obtain target scan data and target control data, and send the target scan data and the target control data to the image reconstruction component 1123 and the second control component 1122, respectively. In some embodiments, the second control component 1122 may control the image reconstruction component 1123 to perform an image reconstruction operation according to the target control data. The image reconstruction component 1123 may perform the image reconstruction operation based on the target scan data to generate the image of the subject.

FIG. 8 is a schematic diagram illustrating an exemplary medical system 800 according to some embodiments of the present disclosure. The data transmission system of the medical system 800 may be configured to transmit data from the stationary part 112 to the rotating part 111. For example, the data transmission system may be configured to transmit a control instruction that is used to cause one or more components of the rotating part 111 to perform one or more operations from the stationary part 112 to the rotating part 111.

In some embodiments, the stationary component 112 may further include a second modulation driving component 1124. The data transmission system may include a second optical source 623 and second photoelectric receiver(s) 624. The second optical source 623 may be mounted on the stationary part 112, and the second photoelectric receiver(s) 624 may be mounted on the rotating part 111.

The second control component 1122 may generate the control instruction, and send the control instruction to the second modulation driving component 1124. In some embodiments, the control instruction may include a plurality of control sub-instructions for causing a plurality of components of the medical device to perform operations. The plurality of control sub-instructions may have different formats and need to be transmitted simultaneously through a same transmission channel. Therefore, the second control component 1122 may send the control instruction to the second conversion component 1121. The second conversion component 1121 may perform a first format conversion on the plurality of control sub-instructions to unify the plurality of control sub-instructions into the same format for subsequent transmission. For example, the plurality of control sub-instructions may be encoded by using a preset encoding algorithm. Then, the second conversion component 1121 may send the plurality of control sub-instructions with the same format to the second modulation driving component 1124. The second modulation driving component 1124 may be configured to drive the second optical source 623 to emit optical signals encoding the control instruction. In some embodiments, before the second modulation driving component 1124 drives the second optical source 623 to emit the optical signals, the second modulation driving component 1124 may modulate the control instruction. Exemplary modulation methods may include an on-off keying (OOK), an amplitude shift keying (ASK), a frequency shift keying (FSK), a phase shift keying (PSK), or the like, or any combination thereof.

The second photoelectric receiver(s) 624 may detect the optical signals from the second optical source 623 and extract the control instruction from the detected optical signals, and send the control instruction to the first conversion component 1114 or the first control component 1113. In some embodiments, the second photoelectric receiver(s) 624 may include a second modulation and demodulation component 6241 configured to modulate and demodulate the detected optical signals to extract the scan data and control data. In some embodiments, each of the second photoelectric receiver(s) 624 may include a modulation and demodulation sub-component configured to modulate and demodulate the optical signals detected by the second photoelectric receiver 624. Exemplary modulation and demodulation operations may include a signal filtering operation, a signal amplifying operation, an analog-to-digital conversion operation, or the like, or any combination thereof. In some embodiments, the first conversion component 1114 may perform a second format conversion on the control instruction to obtain target control instruction, and send the target control instruction to the first control component 1113. The first control component 1113 may control the medical device 110 to perform corresponding operations.

FIG. 9 is a schematic diagram illustrating an exemplary medical system 900 according to some embodiments of the present disclosure. The data transmission system 900 in FIG. 9 may be a combination of the data transmission systems 700 and 800 in FIG. 7 and FIG. 8.

As shown in FIG. 9, the data transmission from the rotating part 111 to the stationary part 112 may be performed along a data transmission link of solid lines shown in FIG. 9 in a similar manner as the transmission of the scan data described in FIG. 7. The data transmission from the stationary part 112 to the rotating part 111 may be performed along a data transmission link of dotted lines shown in FIG. 9 in a similar manner as the transmission of the control instruction described in FIG. 8.

FIG. 10 is a schematic diagram illustrating an exemplary data transmission system 1000 according to some embodiments of the present disclosure.

As shown in FIG. 10, the stationary part 112 may include a first stationary part 112-1 and a second stationary part 112-2. The first stationary part 112-1 and the second stationary part 112-2 may have a shape of a hollow cylinder (as indicated by grey annulus in figures), and the first stationary part 112-1 may be located inside the second stationary part 112-2 to form a scanning cavity of the medical device 110 for accommodating a subject being scanned. The rotating part 111 may be located between the first stationary part 112-1 and the second stationary part 112-2.

The data transmission system 1000 may include the first optical source 621 and one first photoelectric receiver 622. The first optical source 621 and the first photoelectric receiver 622 may transmit data from the rotating part 111 to the stationary part 112 in SISO mode. The first optical source 621 may be mounted on the rotating part 111 and the first photoelectric receiver 622 may be mounted on the stationary part 112. For example, the first optical source 621 may be arranged on a side of the rotating part 111 close to the first stationary part 112-1 along the circumference of the rotating part 111 and form a ring-shaped optical source. The first photoelectric receiver 622 may be mounted on the first stationary part 112-1. In some embodiments, the first optical source 621 may cover the rotating part 111 along a circumferential of the rotating part 111, that is, a length of the first optical source 621 along the circumferential of the rotating part 111 may be equal to a circumference of a cylindrical surface of the rotating part 111 where the first optical source 621 is arranged.

In some embodiments, the first optical source 621 may include first optical emitters configured to emit optical signals encoding target data. The first photoreceiver 622 may receive optical signals in real time. In this case, a modulation driving module (e.g., the first modulation driving module 1115) for driving the first optical source 621 may only have one port to output a signal to drive the first optical source 621 to emit the optical signals. In some embodiments, the modulation driving module may modulate data at a relatively high frequency and control the first optical source 621 on or off. For example, if the first optical source 621 is on (i.e., is emitting light), it may indicate the encoded data is 1 bit, and if the first optical source 621 is off (i.e., is not emitting light), it may indicate the encoded data is 0 bit. When the rotating part 111 rotates during a medical procedure, the first optical source 621 may rotate with the rotating part 111, so that at least one of the first optical emitters may move into a detection zone of the first photoelectric receiver 622, and the first photoelectric receiver 622 may detect the optical signals from the at least one first optical emitter (or the portion of the first light strip) and extract the target data from the detected optical signals.

In some embodiments, the data transmission system 1000 may also include the second optical source 623 and one second photoelectric receiver 624. The second optical source 623 may be similar to the first optical source 621 except that the second optical source 623 is mounted on the stationary part 112 (e.g., on a side of the second stationary part 112-2 close to the rotating part 111). The second photoelectric receiver 624 may be similar to the first photoelectric receiver 622 except that the second photoelectric receiver 624 is mounted on the rotating part 111 (e.g., on a side of the rotating part 111 close to the second stationary part 112-2). In other words, the second optical source 623 may remain still during the medical procedure, while the second photoelectric receiver 624 may rotate during the medical procedure.

FIG. 11 is a schematic diagram illustrating an exemplary data transmission system 1100 according to some embodiments of the present disclosure. The data transmission system 1100 in FIG. 11 may be similar to the data transmission system 1000 in FIG. 10, except for certain components or features.

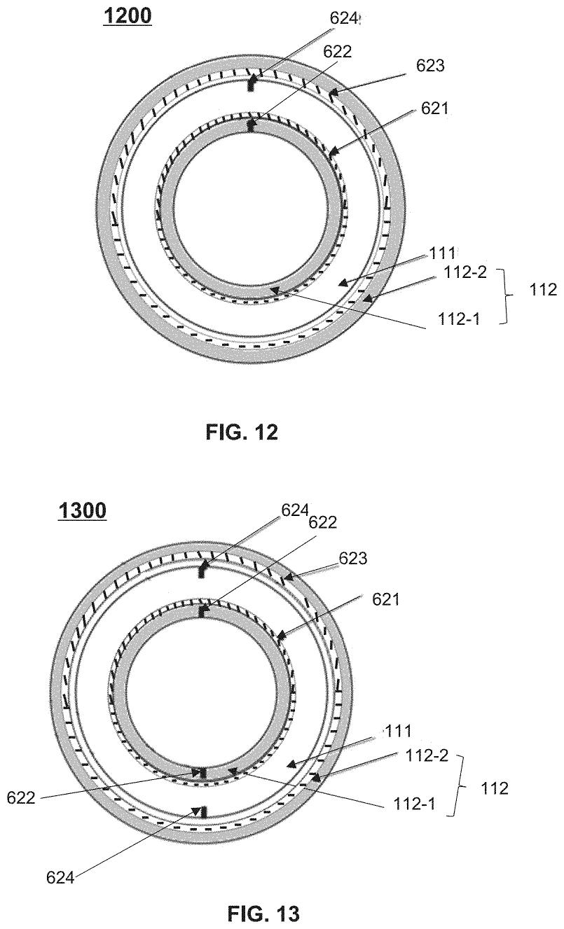

As shown in FIG. 11, the data transmission system 620 may include the first optical source 621 and a plurality of first photoelectric receivers 622 (e.g., two second photoelectric receivers 622 shown in FIG. 12). The first optical source 621 and the plurality of first photoelectric receivers 622 may transmit data from the rotating part 111 to the stationary part 112 in SIMO mode. The first optical source 621 in FIG .11 may be similar to the first optical source 621 in FIG. 10, and the difference is that the first optical source 621 in FIG. 11 only cover a portion of the rotating part 111 along the circumferential of the rotating part 111. A length L1 of the first optical source 621 along the circumferential of the rotating part 111 may be determined by dividing a circumference C1 of a cylindrical surface of the rotating part 111 where the first optical source 621 is arranged by a count N1 of the plurality of first photoelectric receivers 622, that is, L1=C1/N1.

When the rotating part 111 rotates during a medical procedure, the first optical source 621 may rotate with the rotating part 111, so that the plurality of first photoelectric receivers 622 may sequentially detect the optical signals from the first optical source 621 and extract the target data from the detected optical signals. Merely by way of example, when the rotating part 111 rotates from 0 degree to 180 degrees, optical signals may be detected by the first photoelectric receiver 622 on the upper portion of the stationary part 112-1; when the rotating part 111 rotates from 180 degrees to 360 degrees, optical signals may be detected by the first photoelectric receiver 622 on the bottom portion of the stationary part 112-1. During the rotation of the rotating part 111, there is always one first photoelectric receiver 1122 can detect optical signals from the first optical source 621.

In some embodiments, during the rotation of the rotating part 111, angular position information of the rotating part 111 may be obtained through an angle encoder. A modulation and demodulation module may obtain the detected optical signals from the corresponding first photoelectric receiver 622 according to the angular position information of the rotating part 111, and modulate and demodulate the detected optical signals to extract the target data.

In this way, the length of first optical source 621 may decrease as the count of the plurality of first photoelectric receivers 622 increases. Further, if the length of first optical source 621 is relatively small, the modulation speed of the transmitted data may be relatively high, thereby improving the efficiency of data transmission.

In some embodiments, the data transmission system 1100 may also include the second optical source 623 and a plurality of second photoelectric receivers 624 (e.g., two second photoelectric receivers 624 shown in FIG. 12). The second optical source 623 may be similar to the first optical source 621 of the data transmission system 1100 except that the second optical source 623 is mounted on the stationary part 112 (e.g., on a side of the second stationary part 112-2 close to the rotating part 111). The second photoelectric receivers 624 may be similar to the first photoelectric receivers 622 data transmission system 1100 except that the second photoelectric receivers 624 are mounted on the rotating part 111 (e.g., on a side of the rotating part 111 close to the second stationary part 112-2). In other words, the second optical source 623 may remain still during the medical procedure, while the second photoelectric receivers 624 may rotate during the medical procedure.

FIG. 12 is a schematic diagram illustrating an exemplary data transmission system 1200 in MISO mode according to some embodiments of the present disclosure.

As shown in FIG. 12, the data transmission system 1200 may include the first optical source 621 and one first photoelectric receiver 622. The first optical source 621 and the first photoelectric receiver 622 may transmit data from the rotating part 111 to the stationary part 112 in MISO mode. The first optical source 621 and the first photoelectric receiver 622 may be similar to the first optical source 621 and the first photoelectric receiver 622 as described in FIG. 10, except that the first optical source 621 includes a plurality of first optical sub-arrays (e.g., a vertical line shading portion and a straight line shading portion shown in FIG. 12).

During the rotation of the rotating part 111, only one first optical sub-array that moves into a detection zone of the photoelectric receiver 622 may be driven to emit optical signals. For example, during the rotation of the rotating part 111, angular position information of the rotating part 111 may be obtained through an angle encoder, and the first optical sub-array that moves into the detection zone of the photoelectric receiver 622 at the current time may be determined according to the angular position information of the rotating part 1111. A modulation driving module may drive the first optical sub-array that moves into the detection zone of the photoelectric receiver 622 at the current time. In this way, the modulation driving module may sequentially drive the plurality of first optical sub-arrays, which may reduce the heat generated by the data transmission system 1200. Moreover, the length of the first optical sub-array driven each time is relatively small, the modulation speed may be relatively high, thereby improving the efficiency of the data transmission.

In some embodiments, the data transmission system 1200 may also include the second optical source 623 and one second photoelectric receiver 624. For example, the second optical source 623 and the second photoelectric receiver 624 may transmit data from the stationary part 112 to the rotating part 111 in MISO mode. As shown in FIG. 12, the second optical source 623 in FIG .12 may be similar to the second optical source 623 in FIG .10, and the difference is that the second optical source 623 includes a plurality of second optical sub-arrays (e.g., a vertical line shading portion and a straight line shading portion shown in FIG. 12). The second optical source 623 and the second photoelectric receiver 624 may transmit data in a similar manner as how the first optical source 621 and the first photoelectric receiver 622 transmit data.

FIG. 13 is a schematic diagram illustrating an exemplary data transmission system 1300 in MIMO mode according to some embodiments of the present disclosure.

As shown in FIG. 13, the data transmission system 1300 may include the first optical source 621 and a plurality of first photoelectric receivers 622 (e.g., two first photoelectric receivers 622 shown in FIG. 13). The first optical source 621 and the plurality of first photoelectric receivers 622 may transmit data from the rotating part 111 to the stationary part 112 in MIMO mode. The first optical source 621 in FIG. 13 may be the same as or similar to the first optical source 621 in FIG. 12. Each of the plurality of first photoelectric receivers 622 in FIG. 13 may be the same as or similar to the first photoelectric receiver 622 in FIG. 12.

During transmission of target data, the target data may be divided into a plurality sets of data. The first optical source 621 may include a plurality of first optical sub-arrays (e.g., a vertical line shading portion and a straight line shading portion shown in FIG. 13). In this case, a modulation driving module (e.g., the first modulation driving module 1115) for driving the first optical source 621 may have a plurality of ports to output a plurality of signals to simultaneously drive the plurality of first optical sub-arrays to emit optical signals, and the plurality of first photoelectric receivers 622 may detect the optical signals from the plurality of first optical sub-arrays at the same time. In this way, the target data may be transmitted via a plurality of transmission links, simultaneously, which may greatly improve the efficiency of the data transmission.

In some embodiments, each of the plurality of first optical sub-arrays may be configured to transmit one set of the plurality sets of data. When the rotating part 111 rotates during a medical procedure for obtaining the target data, the optical signals from each of the plurality of first optical sub-arrays may be detected by one of the plurality of first photoelectric receiver 622. In some embodiments, a count of the plurality of first optical sub-arrays may be according to a transmission speed of the target data. If the transmission speed of the target data is relatively great, the count of the plurality of first optical sub-arrays may be relatively great.

In some embodiments, each of the plurality of first photoelectric receiver 622 may be configured to receive one set of the plurality sets of data. When the rotating part 111 rotates during the medical procedure, for each of the plurality of first photoelectric receiver 622, one of the plurality of first optical sub-arrays may move into a detection zone of the photoelectric receiver 622 and be driven to emit optical signals, and the first photoelectric receiver 622 may detect the optical signals from the first optical sub-array. In some embodiments, a count of the plurality of first photoelectric receiver 622 may be according to the transmission speed of the target data. If the transmission speed of the target data is relatively great, the count of the plurality of first photoelectric receiver 622 may be relatively great.

In some embodiments, the data transmission system 1300 may also include the second optical source 623 and a plurality of second photoelectric receivers 624 (e.g., two second photoelectric receivers 624 shown in FIG. 13). For example, the second optical source 623 and the second photoelectric receivers 624 may transmit data from the stationary part 112 to the rotating part 111 in MIMO mode. The second optical source 623 in FIG. 13 may be the same as or similar to the second optical source 623 in FIG. 12. Each of the plurality of second photoelectric receivers 624 in FIG. 13 may be the same as or similar to the second photoelectric receiver 624 in FIG. 12. The second optical source 623 and the plurality of second photoelectric receivers 624 may transmit data in a similar manner as how the first optical source 621 and the plurality of first photoelectric receivers 622 transmit data.

It should be noted that the examples in FIGS. 10-13 are provided for illustration purposes, and can be modified according to actual needs. For example, the data transmission mode from the rotating part 111 to the stationary part 112 may be different from the data transmission mode from the stationary part 112 to the rotating part 111. Merely by way of example, data may be transmitted from the rotating part 111 to the stationary part 112 via the SIMO mode (i.e., using an optical source covering a portion of the circumferential of the rotating part 111 and multiple optical receivers mounted on the stationary part 112), while data may be transmitted from the stationary part 112 to the rotating part 111 via the MIMO mode (i.e., using a plurality of optical sub-arrays mounted on the stationary part 112 and a plurality of optical receivers mounted on the rotating part 111).

It should be noted that the first optical source 621 and/or the second optical source 623 in the form of optical array are merely provided for illustration purposes, and not intended to limit the scope of the present disclosure. In some embodiments, the first optical source 621 (or the second optical source 623) may include other components that have the same function as an optical array. For illustration purposes, exemplary components of the first optical source 621 is described hereinafter. The first optical source 621 may include one or more optical emitters and a leaky optical waveguide media (e.g., a planar waveguide media, a side-lighting optical fiber, a leaky optical fiber, etc.). The leaky optical waveguide media may be configured to transmit optical signals emitted by the one or more optical emitters. When the rotating part 111 rotates during the medical procedure, at least a portion of the leaky optical waveguide media moves into the detection zone of the one or more photoelectric receivers, and the one or more photoelectric receivers may detect the optical signals from at least the portion of the leaky optical waveguide media and extract the target data from the detected optical signals.

In some embodiments, the leaky optical waveguide media may be a form of light strip. The position of the aforementioned first optical source 621 in the form of optical array may be replaced with the leaky optical waveguide media. For example, in SISO mode or SIMO mode, the first optical source 621 may include a complete light strip, that is, the light strip includes only one continuous light segment. The light strip may be arranged on the rotating part 111 along the circumference of the rotating part 111 and form a ring-shaped light strip. As another example, in MISO mode or MIMO mode, the first optical source 621 may include a light strip that includes a plurality of light segments. Each of the plurality of light segments may transmit optical signals emitted by one optical emitter. In some embodiments, the plurality of light segments may be arranged on the rotating part 111 along the circumference of the rotating part 111 and form one ring-shaped light strip. Alternatively or optionally, the plurality of light segments may be arranged on the rotating part 111 along the circumference of the rotating part 111 and form a plurality of ring-shaped light strips.

In some embodiments, the leaky optical waveguide media may transmit optical signals using a wavelength division multiplexing (WDM) technology. Specifically, a light segment may simultaneously transmit optical signals with different wavelengths, and the optical signals with different wavelengths may be detected by different photoelectric receivers.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A data transmission system used in a medical device, wherein
the medical device includes a rotating part that rotates during a medical procedure and a stationary part that keeps still during the medical procedure,
the data transmission system includes a radio frequency (RF) emitter and an RF receiver, the RF emitter being mounted on one of the rotating part and the stationary part, the RF receiver being mounted on the other one of the rotating part and the stationary part,
the RF emitter is configured to generate a target RF signal encoding target data based on a current relative position of the RF emitter with respect to the RF receiver, and transmit the target RF signal to the RF receiver,
the RF receiver is configured to receive the target RF signal and extract the target data from the target RF signal.

2. The data transmission system of claim 1, wherein
the RF emitter is mounted on the rotating part,
the RF receiver is mounted on the stationary part, and
the target data includes scan data of a subject collected during the medical procedure.

3. The data transmission system of claim 2, wherein the RF receiver is further configured to transmit the scan data extracted from the target RF signal to an image reconstruction component for reconstructing an image of the subject based on the scan data.

4. The data transmission system of claim 1, wherein the RF emitter includes a plurality of emitting units, and the target RF signal is generated by perform operations including:
generating an initial RF signal including a plurality of initial RF sub-signals each of which is generated via one of the plurality of emitting units of the RF emitter; and
for each of the plurality of initial RF sub-signals,
generating a target RF sub-signal by adjusting one or more parameters of the initial RF sub-signal based on the current relative position of the RF emitter with respect to the RF receiver; and
generating the target RF signal based on the plurality of target RF sub-signals.

5. The data transmission system of claim 4, wherein the one or more parameters include one or more of a carrier frequency, an encoding mode, a modulation mode, a magnitude, or a phase.

6. The data transmission system of claim 5, wherein the adjusting one or more parameters of the initial RF sub-signal based on the current relative position of the RF emitter with respect to the RF receiver includes:
for each of the one or more parameters of the initial RF sub-signal, obtaining a relationship between an adjustment coefficient of the parameter and a
relative position of the RF emitter with respect to the RF receiver; and adjusting the parameter based on the relationship and the current relative position
of the RF emitter with respect to the RF receiver.

7. The data transmission system of claim 6, wherein the relationship between an adjustment coefficient of a parameter of the initial RF sub-signal and the relative position is determined by:
determining an initial relationship between the adjustment coefficient of the parameter and the relative position; and
generating a test result of the initial relationship by performing a reference medical procedure; and
determining the relationship between the adjustment coefficient of the parameter and the relative position by modifying the initial relationship based on the test result.

8. The data transmission system of claim 7, wherein the determining an initial relationship between the adjustment coefficient of the parameter and the relative position includes:
determining possible relative positions of the RF emitter with respect to the RF receiver during a rotation period of the rotating part;
for each of the possible relative positions, estimating an impact of at least one of a Doppler effect or a multipath effect corresponding to the possible relative position,
for each of the possible relative positions, determining an adjustment value corresponding to the parameter based on the estimated impact; and
determining the initial relationship between the adjustment coefficient of the parameter and the relative position based on the adjustment value corresponding to each of the possible relative positions.

9. The data transmission system of claim 1, wherein the RF receiver is mounted on the stationary part, an installation location of the RF receiver on the stationary part is determined by:
obtaining a three-dimension (3D) model of the medical device, the 3D model including a virtual rotating part, a virtual RF emitter mounted on the virtual rotating part, a virtual stationary part, and a plurality of virtual RF receivers mounted on a plurality of reference installation positions of the virtual stationary part,
for each of the plurality of reference installation positions, determining an evaluation result corresponding to the reference installation position by simulating a data transmission process between the virtual RF emitter and the virtual RF receiver mounted on the reference install position; and
determining, based on the evaluation results of the plurality of reference installation positions, the installation location of the RF receiver on the stationary part.

10. A data transmission system used in a medical device, wherein
the medical device includes a rotating part that rotates during a medical procedure and a stationary part that keeps still during the medical procedure,
the data transmission system includes an optical source and at least one photoelectric receiver, the optical source being mounted on one of the rotating part and the stationary part, the at least one photoelectric receiver being mounted on the other one of the rotating part and the stationary part,
the optical source includes optical emitters configured to emit optical signals encoding target data,
when the rotating part rotates during the medical procedure, at least one of the optical emitters moves into a detection zone of the at least one photoelectric receiver, and the at least one photoelectric receiver is configured to detect the optical signals from the at least one optical emitter and extract the target data from the detected optical signals.

11. The data transmission system of claim 10, wherein
the optical source is mounted on the rotating part,
the at least one photoelectric receiver is mounted on the stationary part, and
the target data includes scan data of a subject collected during the medical procedure.

12. The data transmission system of claim 10, wherein
the optical source is mounted on the stationary part,
the at least one photoelectric receiver is mounted on the rotating part, and
the target data includes a control instruction that is used to cause one or more components of the rotating part to perform one or more operations.

13. The data transmission system of claim 11, wherein
the optical source covers a portion of the rotating part along a circumferential of the rotating part, and
the at least one photoelectric receiver includes a plurality of photoelectric receivers, when the rotating part rotates during the medical procedure, the plurality of photoelectric receivers sequentially detect the optical signals from the optical source.

14. The data transmission system of claim 11, wherein
the at least one photoelectric receiver includes one photoelectric receiver, and
the optical source covers the rotating part along a circumferential of the rotating part.

15. The data transmission system of claim 11, wherein
the target data is divided into a plurality sets of data,
the optical source includes a plurality of optical sub-arrays each of which is configured to transmit one set of the plurality sets of data,
the at least one photoelectric receiver includes a plurality of photoelectric receivers, when the rotating part rotates during the medical procedure, the optical signals from each of the plurality of optical sub-arrays is detected by one of the plurality of photoelectric receivers.

16. The data transmission system of claim 14, wherein a count of the plurality of optical sub-arrays is determined according to a transmission speed of the target data.

17. The data transmission system of claim 11, wherein the at least one photoelectric receiver is further configured to transmit the scan data extracted from the detected optical signals to an image reconstruction component for reconstructing an image of the subject based on the scan data.

18. The data transmission system of claim 10, wherein the one of the rotating part and the stationary part further includes a modulation driving module configured to drive the optical source to emit the optical signals.

19. The data transmission system of claim 10, wherein the at least one photoelectric receiver further includes a modulation and demodulation module configured to modulate and demodulate the detected optical signals to extract the target data.

20. The data transmission system of claim 10, wherein
the data transmission system includes a second optical source and at least one second photoelectric receiver,
the second optical source is mounted on the other one of the rotating part and the stationary part where the at least one photoelectric receiver is mounted on, and
the at least one second photoelectric receiver is mounted on the one of the rotating part and the stationary part where the optical source is mounted on.
